(19) United States
(12) Patent Application Publication
(10) Pub. No.: US 2007/0106121 A1
(43) Pub. Date: May 10, 2007

(54) INTUBATION ASSISTANCE APPARATUS AND INTUBATION ASSISTANCE USED IN THE APPARATUS

(75) Inventors: Hidetaka Yokota, Tokyo (JP); Yukio Taniguchi, Tokyo (JP); Junichi Koyama, Nagano (JP)

Correspondence Address: GREENBLUM & BERNSTEIN, P.L.C., 1950 ROLAND CLARKE PLACE, RESTON, VA, 20191, US

(73) Assignees: Junichi Koyama, Nagano (JP); PENTAX Corporation, Tokyo (JP)

(21) Appl. No.: 11/552,184

(22) Filed: Oct. 24, 2006

(30) Foreign Application Priority Data
Oct. 24, 2005 (JP) P2005-309150
Oct. 24, 2005 (JP) P2005-309151

(51) Int. Cl. A61B 1/267; A61B 1/04

(52) U.S. Cl. 600/188; 600/120; 600/191; 600/179

(57) ABSTRACT

An intubation assistance apparatus includes a main body and an intubation assistance instrument detachably mounted to the main body. The intubation assistance instrument has an elongated insertion section for insertion into the trachea or its vicinity of a patient from the mouth. The insertion section of the intubation assistance instrument is provided with a groove for leading an intubation tube to the trachea of the patient and a scope guide bore for receiving a laryngoscope with a CCD and a white LED. A distal end portion of the scope guide bore is fluid-tightly (air-tightly) sealed. The scope guide bore is fluid-tightly (air-tightly) sealed in its entirety in a state that the intubation assistance instrument is mounted to the main body. A plate-like tongue piece protruding forward and having optical transparency is formed on a distal end portion of the insertion section.

### BACKGROUND OF THE INVENTION

1. Field of the Invention

The present invention relates to an intubation assistance apparatus for use in inserting a distal end of an intubation tube into a target site such as a trachea of a patient, and the present invention also relates to an intubation assistance instrument used in the intubation assistance apparatus.

2. Description of the Prior Art

It is sometimes necessary to practice artificial respiration, as a first-aid lifesaving treatment for a patient who is suffering from unconsciousness caused by an accident or the like. Although the artificial respiration may be practiced without having to use any instrument or apparatus, it is often the case that a respirator is used for that purpose.

In the event that a respirator is utilized to conduct artificial respiration, an intubation tube whose proximal end is connected to the respirator is inserted into the trachea of a patient to supply air to the trachea from the respirator via the tube.

In the meantime, if a patient loses consciousness, the root of a tongue is retracted to thereby block up a respiratory tract because of the relaxation of muscles of the pharynx and the larynx and/or the gravity-caused loosening of a lower jaw.

Therefore, in the case where the afore-mentioned intubation tube is to be inserted into the trachea or a target area (which operation will be hereinafter referred to as “intubation operation”), it is essential to first open the blocked respiratory tract and secure an air passage by pulling up the tongue.

As an instrument for use in securing the air passage, there is known what is called an oral airway (see, e.g., JP-A8-322937).

Such an oral airway is an elongated member with a curved distal end and can be inserted through the mouth of a patient who has lost consciousness, for instance, whereby an appropriate portion on the side of the distal end comes into contact with and lifts up a tongue root portion of the patient, thus securing the air passage.

However, the oral airway is an instrument merely for securing the air passage and, therefore, an operator cannot observe the pharynx or the larynx (and a rima glottidis in the larynx) by use of the oral airway. This means that it is difficult for the operator to perform an intubation operation while the oral airway is in use,

A video laryngoscope is known in the art as an auxiliary intubation instrument for securing an air passage. The video laryngoscope includes an insertion section having a pinch bar-like shape and used in securing the air passage, an image pickup device, such as a CCD or the like, provided on the distal end of the insertion section, and an image display means, such as a display or the like, for displaying an image taken by the image pickup device.

The video laryngoscope is in possession of both the intubation assistance function of securing the air passage and the function of allowing an operator to observe the pharynx and the larynx.

Use of such a video laryngoscope makes it possible for the operator to observe the pharynx and the larynx, thus facilitating the intubation operation to a certain extent However, a high degree of technical skill is required in inserting an intubation tube made of a flexible material into the trachea through the small rima glottidis.

As noted above, the conventional instruments still require a skilled technique for an operator to carry out the intubation operation in an easy and reliable manner.

Further, in the intubation operation using the video laryngoscope, the intubation tube cannot be inserted unless the jaws of a patient are caused to protrude forward (the cervical spine is bent in advance) to arrange the mouth cavity and the rima glottidis in a generally straight line. Therefore, in case of a patient suffering from a cervical spine contusion (a patient showing the symptoms of intubation trouble), it is impossible to bend the cervical spine, and thus resulting in a problem in that the video laryngoscope is no longer usable.

Furthermore, the insertion section of the video laryngoscope to be inserted into the mouth of a patient is integrally provided with a main body thereof. Therefore, even if cleansing, disinfecting and sterilizing operations are carried out for the entire instrument every time upon its use, it is undesirable to repeatedly use the same laryngoscope to other patient from the viewpoint of safety such as a need for preventing the patient from being infected with bacteria and the like.

In addition, since a hole in the distal end portion of the insertion section in which the image pickup device is placed is not sealed, it is necessary to perform cleansing, disinfecting and sterilizing operations for the entire instrument including both the outside and inside of the insertion section thereof every time upon the use of the laryngoscope. This causes a drawback in that it takes time and trouble to perform such operations.

### SUMMARY OF THE INVENTION

Accordingly, a main object of the present invention is to provide an intubation assistance apparatus by which an intubation operation can be carried out easily and reliably with simple operation as well as increased safety and which can reduce the labor hour when it is used.

Further, another object of the present invention is to provide an intubation assistance instrument used in the intubation assistance apparatus.

In order to achieve the main object, the present invention is directed to an intubation assistance apparatus which includes a main body; an intubation assistance instrument provided on the main body, the intubation assistance instrument having an elongated insertion section for insertion into a target site of a patient from a mouth cavity or a nasal cavity of the patient; and image light acquiring means for acquiring image light of an observation site at a distal end portion of the insertion section. The intubation assistance instrument includes guide means provided on the insertion section for leading an intubation tube to the target site of the patient when the intubation tube is inserted into the target site, in which the intubation tube is adapted to be removed from the guide means in a state that the insertion section is kept inserted into the target site; an internal bore in which at least a part of the image light acquiring means is disposed; and a plate-like protruding portion provided on a distal end portion of the insertion section so as to protrude in a frontward direction.

According to the present invention described above, it is possible to provide an intubation assistance apparatus by which an intubation operation can be carried out easily and reliably with simple operation and increased safety.

Particularly, provision of the guide means eliminates the need for a patient to take, during the time of inserting the intubation tube into the trachea thereof, a posture in which the cervical spine of the patient is bent to have the jaws protruded forward. Accordingly, the intubation operation can be performed easily and reliably even for a patient suffering from a cervical spine contusion (a patient showing the symptoms of intubation trouble).

Further, provision of the plate-like protruding portion at the distal end portion of the insertion section enables the operator to lift up the epiglottis by use of the protruding portion, thereby making it possible to easily and reliably secure an air passage for the patient.

Furthermore, due to the provision of the image light acquiring means, the operator can perform the intubation operation while observing the area around the distal end portion of the insertion section and the positional relationship between the rima glottidis, which is an entrance of the trachea, and the distal end portion of the intubation tube. This allows the intubation operation to be performed easily and reliably.

In the intubation assistance apparatus described above, it is preferred that the target site is a trachea or its vicinity of the patient, and the protruding portion has optical transparency.

Since the protruding portion of the distal end portion of the insertion section is optically transparent, the epiglottis of a patient can be observed through the protruding portion at the time when the epiglottis is lifted up with the protruding portion, thereby making it possible for the operator to lift up the epiglottis in an easy and reliable manner.

Further, in the intubation assistance apparatus described above, it is also preferred that the protruding portion is formed into a generally rectangular shape having a projecting length in the range of 10 to 40 mm.

This also makes it possible for the operator to lift up the epiglottis in an easy and reliable manner.

Further, in the intubation assistance apparatus described above, it is also preferred that the insertion section is curved at a roughly middle part thereof to have an inner surface at the distal end portion of the insertion section, in which the protruding portion is formed straight to continuously extend from the inner surface of the insertion section.

This enables the operator to perform the intubation operation more easily.

Further, in the intubation assistance apparatus described above, it is also preferred that the insertion section has at least one lumen extending along a longitudinal direction of the insertion section.

This makes it possible to dispose a suction tube, a forceps or the like inside the lumen. Use of the suction tube makes it possible to suck up and remove flowable foreign materials inclusive of spit, sputum and so forth. Solid foreign materials can be removed using the forceps.

Furthermore, in the intubation assistance apparatus described above, it is also preferred that the guide means is comprised of a groove extending along the longitudinal direction of the insertion section.

This allows the intubation tube to be guided in a precise and accurate manner, thereby making it possible to perform the intubation operation with increased ease.

Further, in the intubation assistance apparatus described above, it is also preferred that the intubation assistance instrument is detachably mounted to the main body.

This ensures that, by changing the intubation assistance instrument on a use time basis, the need to perform cleansing, disinfecting and sterilizing operations can be eliminated so as to reduce the labor hour.

Furthermore, in the intubation assistance apparatus described above, it is also preferred that the part of the image light acquiring means disposed in the internal bore is removable from the insertion section.

This makes it possible to change only the intubation assistance instrument, with the image light acquiring means being left on the main body.

Furthermore, in the intubation assistance apparatus described above, it is also preferred that the image light acquiring means has a center line of visual field inclined with respect to a center line of the distal end portion of the insertion section in such a manner as to head for the intubation tube.

This allows the operator to quite easily ascertain, during the intubation operation, the positional relationship between the rima glottides and the distal end portion of the intubation tube based on the image light acquired by the image light acquiring means.

Furthermore, in the intubation assistance apparatus described above, it is also preferred that the intubation assistance apparatus further includes image displaying means provided on the main body for displaying an image corresponding to the image light acquired by the image light acquiring means.

This enables the operator to easily ascertain the image light acquired by the image light acquiring means, thereby making it possible to perform the intubation operation in an easy and reliable manner.

Further, in the intubation assistance apparatus described above, it is preferred that the intubation assistance apparatus is configured such that, when the intubation tube is pushed forward from the distal end portion of the insertion section, the intubation tube can be moved toward substantially a center of the image displayed on the image displaying means.

This allows the operator to quite easily ascertain, during the intubation operation, the positional relationship between the rima glottidis and the distal end portion of the intubation tube, by seeing the image displayed on the image displaying means.

Furthermore, in the intubation assistance apparatus described above, it is also preferred that at least a distal end portion of the internal bore is fluid-tightly sealed so that at least a distal end portion of the image light acquiring means is not exposed to the outside from the bore.

This makes it possible to provide an intubation assistance apparatus by which an intubation operation can be carried out easily and reliably with simple operation and increased safety. In particular, since at least the distal end portion of the internal bore in which at least a part of the image light acquiring means is disposed is fluid-tightly sealed, it is not necessary to perform cleansing, disinfecting and sterilizing operations for the image light acquiring means every time upon its use, thus reducing the labor hour.

Furthermore, in the intubation assistance apparatus described above, it is also preferred that the blockage portion has optical transparency.

This makes it possible not to impair the function of the image light acquiring means.

Furthermore, in the intubation assistance apparatus described above, it is also preferred that the intubation assistance apparatus further includes sealing means for fluid-tightly sealing a gap between the main body and the intubation assistance instrument in a state that the intubation assistance instrument is mounted to the main body wherein a proximal end side of the internal bore is fluid-tightly sealed by means of the sealing means.

This makes it possible to prevent fluid (e.g., spit) from infiltrating into the bore at the distal end portion of the insertion section.

Furthermore, in the intubation assistance apparatus described above, it is also preferred that the internal bore is fluid-tightly sealed in its substantially entirety in a state that the intubation assistance instrument is mounted (attached) to the main body.

This makes it possible to prevent fluid (e.g., spit) from infiltrating into the bore at the distal end portion of the insertion section more reliably.

Furthermore, in the intubation assistance apparatus described above, it is also preferred that the intubation assistance apparatus further includes an illumination means for illuminating the observation site, and at least a part of the illumination means is provided in the insertion section so that it can be removed from the insertion section.

This allows the image light acquiring means to acquire clear image light. Further, this also makes it possible to change only the intubation assistance instrument, with the illumination means being left on the main body as well as to prevent any contamination of the illumination means when using the apparatus.

Furthermore, in the intubation assistance apparatus described above, it is also preferred that the illumination means has a light source provided within the distal end portion of the insertion section.

This enables the image light acquiring means to have a simplified structure.

Alternatively, in the intubation assistance apparatus described above, it is preferred that the illumination means has a light source provided within the main body and a light guide means for leading a light from the light source to the distal end portion of the insertion section.

According to this, it becomes possible to use an arbitrary light source without making the size of the insertion section larger.

Moreover, in the intubation assistance apparatus described above, it is also preferred that the part of the image light acquiring means disposed within the internal bore is detachable from the insertion section and a part of the illumination means provided within the insertion section is disposed within the internal bore such that a positional relationship between the part of the illumination means and the part of the image light acquiring means is fixed.

This makes et possible to change only the intubation assistance instrument, with the image light acquiring means and the illumination means being left on the main body as well as to prevent any contamination of the image light acquiring means and the illumination means when using the apparatus.

Another aspect of the present invention resides in an intubation assistance apparatus comprising a main body; an intubation assistance instrument adapted to be detachably mounted to the main body, the intubation assistance instrument having an elongated insertion section for insertion into a target site of a patient from a mouth cavity or a nasal cavity of the patient; and image light acquiring means for acquiring image light of an observation site at a distal end portion of the insertion section, wherein the intubation assistance instrument includes guide means provided on the insertion section for leading an intubation tube to the target site of the patient when the intubation tube is inserted into the target site, in which the intubation tube is adapted to be removed from the guide means in a state that the insertion section is kept inserted into the target site; an internal bore having a disposing portion in which at least a part of the image light acquiring means is adapted to be disposed, the internal bore being formed along a longitudinal direction of the insertion section; and a plate-like protruding portion provided on a distal end portion of the insertion section so as to protrude in a frontward direction, wherein the protruding portion has optical transparency.

According to the invention described above, since the intubation assistance instrument is detachably mounted to the main body, it becomes possible to change the intubation assistance instrument each time when the latter is in use and to thereby keep the patient from being infected (secondarily infected) by bacteria, which helps to enhance the safety.

These and other objects, structures and results of the present invention will be apparent more clearly when the following detailed description of the preferred embodiments is considered taken in conjunction with the appended drawings.

### BRIEF DESCRIPTION OF THE DRAWINGS

FIG. 1 is a perspective view showing an embodiment of an intubation assistance apparatus provided with an intubation assistance instrument according to the present invention.

FIG. 2 is a perspective view illustrating a main body of the intubation assistance apparatus shown in FIG. 1.

FIG. 3 is a perspective view depicting the intubation assistance instrument of the intubation assistance apparatus shown in FIG. 1.

FIG. 4 is another perspective view showing the intubation assistance instrument of the intubation assistance apparatus shown in FIG. 1.

FIG. 5 is a cress-sectional view illustrating a connection portion (coupling portion) of the main body and the intubation assistance instrument of the intubation assistance apparatus shown in FIG. 1.

FIG. 6 is a side and front view showing a laryngoscope of the intubation assistance apparatus shown in FIG. 1.

FIG. 7 is a rear view illustrating a distal end of the intubation assistance instrument of the intubation assistance apparatus shown in FIG. 1.

### DETAILED DESCRIPTION OF THE PREFERRED EMBODIMENT

Hereinbelow, a preferred embodiment of an intubation assistance apparatus according to the present invention will be described in detail with reference to the accompanying drawings.

In the present embodiment, a description will be made with regard to the case where the intubation assistance apparatus of the present invention is used for inserting an intubation tube into a trachea of a patient.

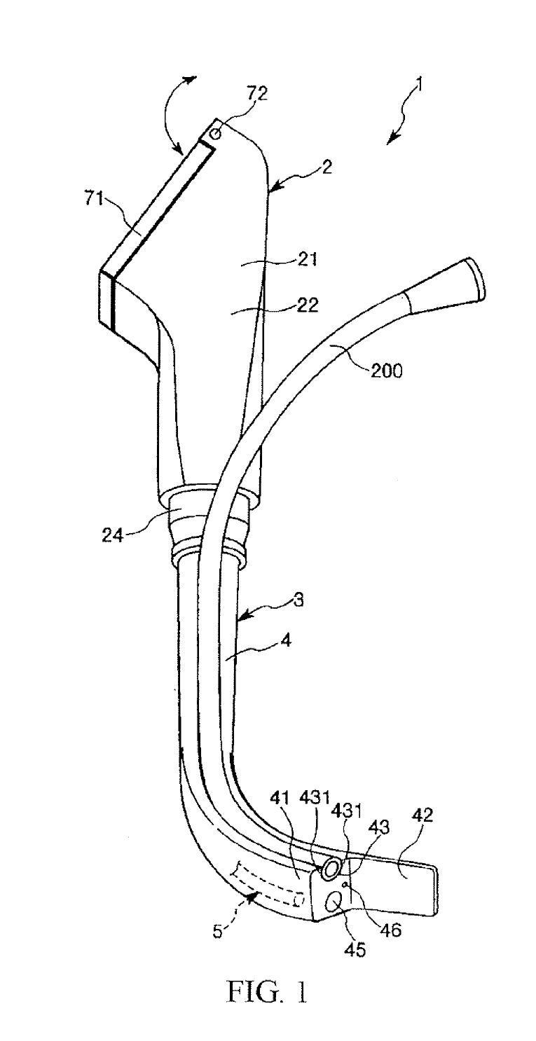

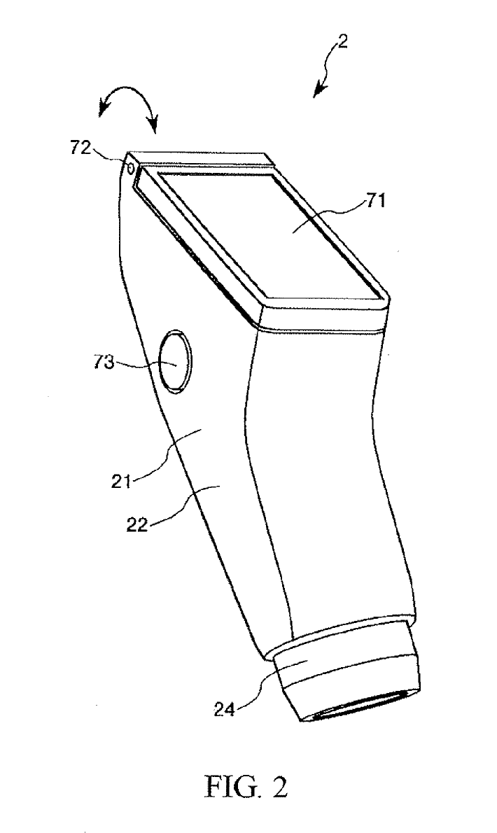

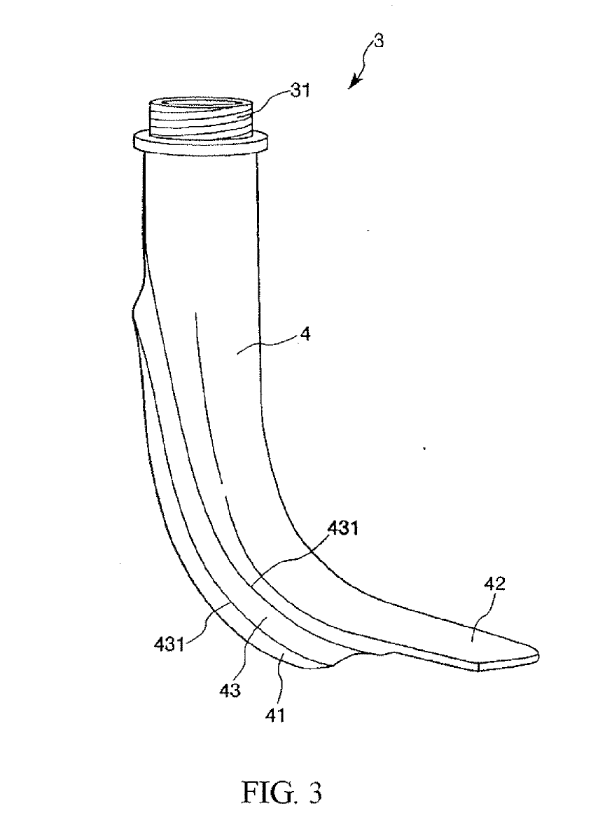

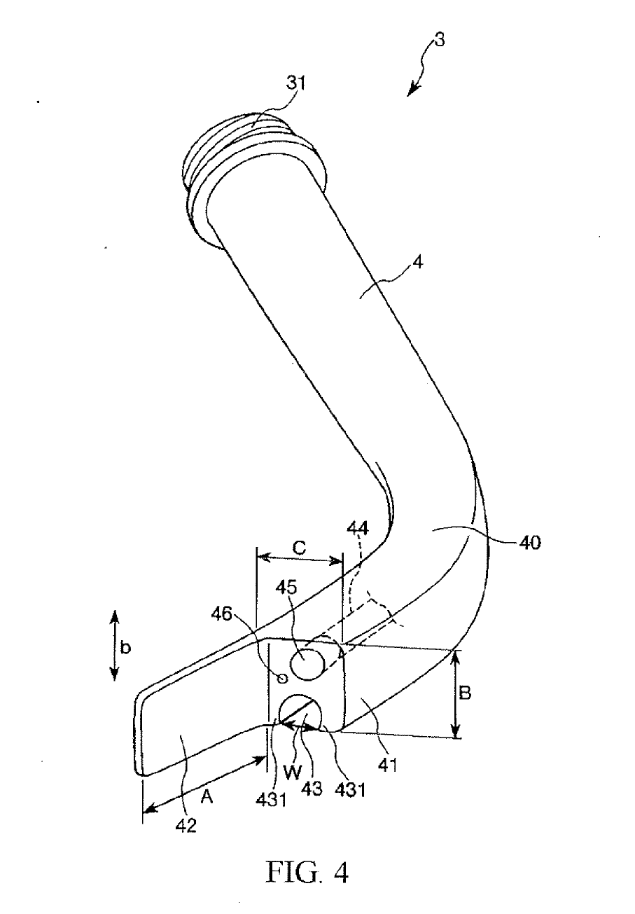

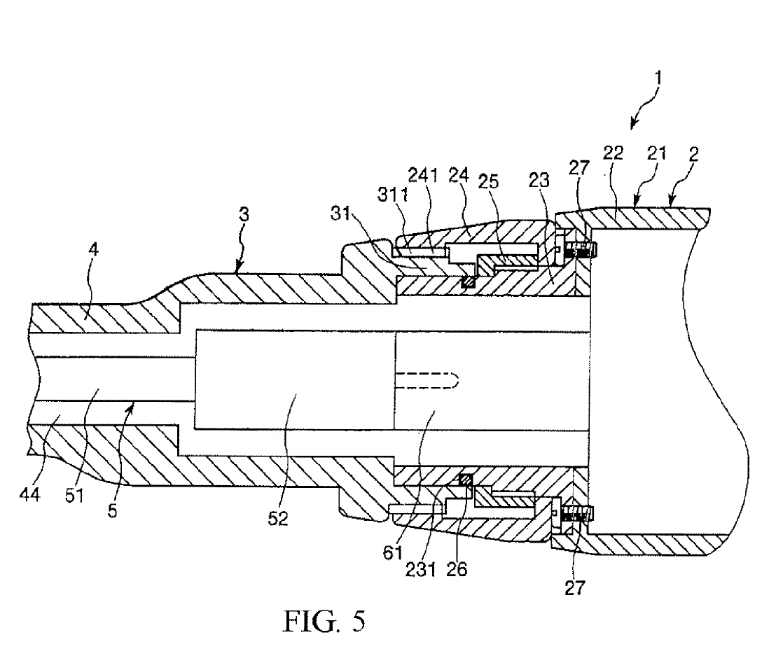

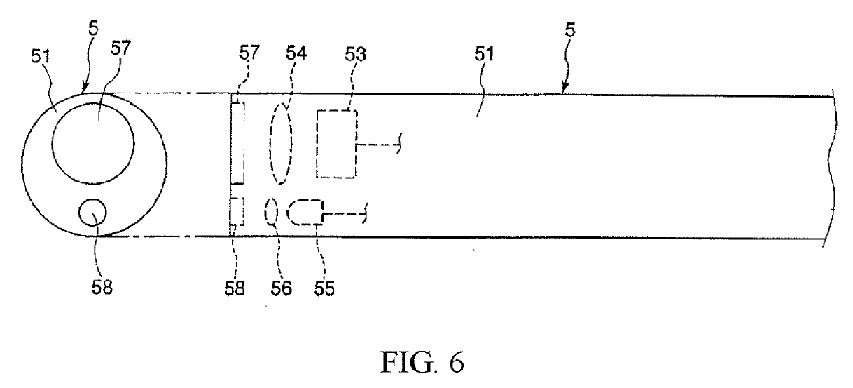

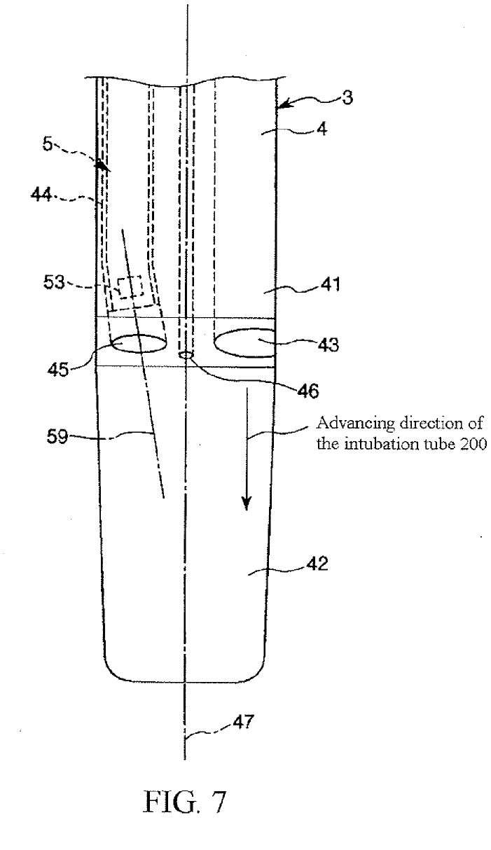

FIG. 1 is a perspective view showing an embodiment of an intubation assistance apparatus provided with an intubation assistance instrument according to the present invention, FIG. 2 is a perspective view illustrating a main body of the intubation assistance apparatus shown in FIG. 1, FIGS. 3 and 4 are perspective views depicting the intubation assistance instrument of the intubation assistance apparatus shown in FIG. 1, FIG. 5 is a sectional view illustrating a connection portion (coupling portion) of the main body and the intubation assistance instrument of the intubation assistance apparatus shown in FIG. 1, FIG. 6 is a side and front view showing a laryngoscope of the intubation assistance apparatus shown in FIG. 1, and FIG. 7 is a rear view illustrating a distal end of the intubation assistance instrument of the intubation assistance apparatus shown in FIG. 1.

In the following description, the lower side and the upper side in FIGS. 1, 3, 4 and 7 will be referred to as “distal end” and “proximal end”, respectively, for the purpose of clarity. However, it should be noted that the direction of the distal end varies in the drawings because the insertion section of the intubation assistance instrument is curved in its midway portion. The left side and the right side in FIGS. 5 and 6 will be referred to as “distal end” and “proximal end”, respectively.

The intubation assistance apparatus 1 shown in these drawings includes a main body 2 and an intubation assistance instrument 3 detachably mounted to the main body 2.

As will be described later, the intubation assistance apparatus 1 is used in combination with an intubation tube 200 which is to be inserted into the trachea of a patient through the mouth (mouth cavity) thereof.

As shown in FIG. 1, the intubation tube 200 has a substantially circular cross-section and is formed of a flexible or pliable material such as elastomer, rubber, and the like.

The intubation assistance instrument 3 is coupled to the main body 2 and is provided with an insertion section 4 that has various additional functions set forth below as well as the function equivalent to that of a typical oral airway.

The insertion section 4 is formed of an elongated member and can be inserted into a target area, namely, the trachea of a patient or its vicinity, from the mouth (mouth cavity) of the patient. By way of example, the insertion section 4 is used in such a manner that it is inserted through the mouth of a patient who has lost consciousness or who is under general anesthesia. An air passage for the patient is secured by bringing an appropriate portion on the side of a distal end of the insertion section 4 into contact with the root of a tongue of the patient, while lifting up the epiglottis of the patient by use of a below-mentioned tongue piece (protruding portion) 42 formed on the side of the distal end of the insertion section 4.

As illustrated in FIGS. 3 and 4, the insertion section 4 is curved at its longitudinal midway part so that the distal end side extension thereof can be oriented upwardly in FIG. 3, thereby forming a curved portion 40. The proximal end side extension of the curved portion 40 makes a generally right angle with respect to the distal end side extension thereof. In the present embodiment, the insertion section 4 (the intubation assistance instrument 3) has optical transparency in its entirety. Alternatively, only a required portion (area) of the insertion section 4 may be optically transparent.

On the left side surface of the insertion section 4 in FIG. 3, a groove (guide means) 43 is provided so as to extend from the proximal end portion to the distal end portion 41 of the insertion section 4, namely along the longitudinal direction of the insertion section 4. In a general method of using the intubation assistance apparatus 1, an operator initially takes a position on the upper side of the head of a lying-down patient and, subsequently, the intubation assistance apparatus 1 is placed so that a display 71 (which will be described later in detail) provided in the intubation assistance apparatus 1 can face the operator. If the operator, the patient and the intubation assistance apparatus 1 are in such a positional relationship (namely, an in-use positional relationship), the groove 43 lies on the right side of the insertion section 4 when viewed from the operator.

Under the state that the air passage is secured by the insertion section 4, the groove 43 serves to guide the intubation tube 200 toward the trachea of the patient as it is inserted through the mouth of the patient. Owing to the fact that the groove 43 is employed as a guide means, it is possible to remove the intubation tube 200 with ease while the insertion section 4 remains inserted into the body of the patient.

Once the air passage for the patient is secured by the insertion section 4, the intubation tube 200 is introduced into the groove 43 at the proximal end portion of the insert ion section 4 and then continues to be pushed toward the distal end portion 41 of the insertion section 4. At this time, the intubation tube 200 is guided by at least the side wall of the groove 43 (by all of the inner walls of the groove 43 in the present embodiment) and is moved forward while making sliding contact with the groove 43. Then the distal end of the intubation tube 200 continues to be moved toward the rima glottidis in the back of the larynx beyond the distal end portion 41 of the insertion section 4. In this process, the groove 43 lies on the right side of the insertion section 4 when viewed from the operator and therefore the operator can operate the intubation tube 200 with his or her right hand in the same manner as is done in the video laryngoscope.

The groove 43 has a cross-section of a generally semicircular shape. The width (maximum width), i e., diameter of the groove 43 is set to have a dimension slightly greater than the outer diameter of the intubation tube 200. The cross-section of the groove 43 is not restricted to the semicircular shape, but may have other shapes such as a U-like shape, a square bracket shape or the like. Furthermore, the guide means is not restricted to a groove, but may be a bore for instance.

On the edges of the left side wall and the right side wall of the groove 43 in FIG. 4, ribs (juts) 431 serving as a stopper (removal prevention means)for the intubation tube 200 are formed so as to extend over the entire length of the groove 43. The rib 431 provided on the edge of the left side wall protrudes toward the edge of the right sidewall, whereas the rib 431 provided on the edge of the right side wall protrudes toward the edge of the left side wall. In other words, one of the ribs 431 is projected toward the other.

Presence of the ribs 431 ensures that the width of the opening portion of the groove 43 (the length “W” in FIG. 4) becomes smaller than the width of the inner portion of the groove 43 than the opening portion. In other words, the width of the opening portion of the groove 43 is smaller than the outer diameter of the intubation tube 200. This precludes the possibility that the intubation tube 200 inserted into the groove 43 is removed from the groove 43 through the opening portion.

On the distal end portion 41 of the insertion section 4, there is a plate-like tongue piece (protruding portion) 42 that protrudes in the direction of the distal end and has optical transparency. The tongue piece 42 is formed straight at the distal end portion 41 to continuously extend from the inner surface of the curved portion 40. During the course of an intubation operation, the epiglottis of a patient can be lifted up with the tongue piece 42 to thereby secure an air passage for the patient in an easy and reliable manner.

The tongue piece 42 is of a generally rectangular shape when viewed from the top. Moreover, the corner portions of the tongue piece 42 are rounded to improve the safety in the process of insertion. The shape of the tongue piece 42 as seen from the top is not restricted to the rectangular one, but may be of other shapes, e.g., a semi-elliptical shape or a semi-circular shape.

In this regard, the projecting length “A” of the tongue piece 42 shown in FIG. 4 is preferably in the range of about 10 to 40 mm, and more preferably about 20 to 30 mm.

If the length “A” of the tongue piece 42 exceeds the upper limit value, a difficulty is encountered during the intubation operation and the strength of the tongue piece 42 becomes insufficient particularly when the thickness thereof is small.

On the other hand, if the length “A” of the tongue piece 42 is less than the lower limit value, it becomes difficult to lift up the epiglottis of the patient during the intubation operation.

Further, it is preferred that the thickness of the tongue piece 42 is about 1 to 2 mm at its distal end portion and about 3 to 5 mm at its proximal end portion.

If the thickness of the tongue piece 42 exceeds the upper limit value, it becomes difficult to lift up the epiglottis of the patient during the intubation operation.

On the other hand, if the thickness of the tongue piece 42 is less than the lower limit value, the strength of the tongue piece 42 may become insufficient depending on the material of which the tongue piece 42 is made.

The insertion section 4 has a generally rectangular cross-section at the distal end portion 41 thereof. The cross-section of the distal end portion 41 of the insertion section 4 is sized such that the transverse length “B” indicated in FIG. 4 (the length in a direction substantially perpendicular to the thickness direction of the tongue piece 42) is preferably in the range of about 15 to 40 mm and more preferably in the range of about 25 to 30 mm, and the vertical length “C” (the length in a direction parallel to the thickness direction of the tongue piece 42) is preferably in the range of about 10 to 30 mm and more preferably in the range of about 15 to 20 mm.

If the transverse length “B” and the vertical length “C” at the cross-section of the distal end portion 41 of the insertion section 4 exceed the upper limit values, it becomes difficult to insert the insertion section 4 into the air passage of the patient and the burden on the patient is increased during the intubation operation.

On the other hand, if the transverse length “B” and the vertical length “C” are less than the lower limit values, the tongue of the patient is hung down to the side of the distal end portion 41 of the insertion section 4 to thereby narrow the field of view of a below-mentioned CCD 53, at the time when the distal end portion 41 of the insertion section 4 is brought into contact with (or pressed against) the root portion of the tongue during the intubation operation. This is because the tongue piece 42 has a plate-like shape.

The cross-section of the distal end portion 41 of the insertion section 4 is not restricted to the rectangular shape but may have other shapes, e.g., an elliptical shape, a semi-elliptical shape, a circular shape, a semi-circular shape or the like.

Formed on the insertion section 4 is a scope guide bore (internal bore) 44 that extends from the proximal end portion to the distal end portion 41, namely, along the longitudinal direction of the insertion section 4, and serves as a disposing portion on which at least a part (the entirety in the present embodiment) of a below-mentioned laryngoscope (image light acquiring means) 5 is disposed. In this connection, the distal end portion (the entirety in the present embodiment) of the scope guide bore 44 is eccentrically located in the width direction of the tongue piece 42 (in the direction indicated by an arrow “b” in FIG. 4). In other words, if the operator, the patient and the intubation assistance apparatus 1 are in the in-use positional relation set forth above, the scope guide bore 44 is eccentrically located to the left when viewed from the operator.

The scope guide bore 44 has a cross-section of a generally circular shape and is opened at the proximal end portion of the insertion section 4. As will be noted below, the proximal end side of the scope guide bore 44 is fluid-tightly (air-tightly) sealed under the state that the intubation assistance instrument 3 is mounted to the main body 2.

Provided on or secured to the distal end portion of the scope guide bore 44 (the distal end portion 41 of the insertion section 4) is a blockage portion 45 having optical transparency. The distal end portion of the scope guide bore 44 is fluid-tightly (air-tightly) sealed by means of the blockage portion 45. Alternatively, the blockage portion 45 may be integrally formed with the insertion section 4.

As illustrated in FIGS. 1 and 5, a laryngoscope 5 is disposed or received inside the scope guide bore so as to be removable therefrom. In this case, the distal end portion of the laryngoscope 5 is arranged at the distal end portion of the scope guide bore 44. In view of the fact that the distal end portion of the scope guide bore 44 remains sealed by means of the blockage means 45 as described above, the distal end portion of the laryngoscope 5 (an image light acquiring means and an illumination means) disposed or received within the scope guide bore 44 is not exposed to the outside (does not make contact with the ambient air). The laryngoscope 5 is a device that functions as both an image light acquiring means for acquiring the image light of an observation site (an image taking means for taking an image of an object) in front of the distal end portion 41 of the insertion section 4 and an illumination means for illuminating the observation site. The laryngoscope 5 has a water-proof structure which may be, for example, one of the conventional water-proof structures (configurations) known in the art.

As shown in FIGS. 5 and 6, the laryngoscope 5 includes a flexible elongated body portion 51 and a connector portion 52 provided on the proximal end portion of the body portion 51. By virtue of the connector portion 52, the laryngoscope 5 is detachably and mechanically connected to a connector portion 61 of the main body 2 set forth below. As a consequence, the laryngoscope 5 and the main body 2 are electrically connected with each other.

A CCD (image pickup device) 53 and a white LED (light emitting diode) 55 serving as a light source are provided within the distal end portion of the body portion 5l One or more image taking lenses (a set of lenses) including an objective lens 54 are provided at the distal end portion of the CCD 53 of the body portion 51, whereas an illumination lens 56 is arranged at the distal end portion of the white LED 55. In the following, the image taking lens including the objective lens 54 will be simply referred to as “objective lens 54”.

More specifically, two holes opened at their tip ends (not shown in the drawings) are formed on the distal end portion of the body portion 51. The CCD 53 and the objective lens 54 are arranged within one of the holes, the tip end of which is fluid-tightly sealed by means of a window portion 57 having optical transparency. The white LED 55 and the illumination lens 56 are arranged within the other of the holes, the tip end of which is fluid-tightly sealed by means of a window portion 58 having optical transparency. As an alternative, the illumination lens 56 may be omitted and the window portion 58 may play the role of the illumination lens S6. By arranging the CCD 53 and the objective lens 54 in one hole and the white LED 55 and the illumination lens 56 in the other hole and by separately providing the window portions 57 and 58 in the respective holes, the CCD 53 and the objective lens 54 are light-shielded from the white LED 55 and the illumination lens 56, thus preventing the light of the white LED 55 from adversely affecting the CCD 53. The body portion 51 has the function of fixing the positional relationship between the CCD 53 and the objective lens 54, the positional relationship between the white LED 55 and the illumination lens 56, and the positional relationship between the CCD 53 plus the objective lens 54 and the white LED 55 plus the illumination lens 56, respectively.

The control line and the signal line of the CCD 53 as well as the signal line of the white LED 55 extend through the body portion 51 and are respectively connected to the corresponding terminals of the connector portion 52. The image light acquiring means is comprised of the CCD 53 and the objective lens 54, whereas the illumination means is comprised of the white LED 55 and the illumination lens 56 (the window portion 58).

In the laryngoscope 5, the light (image light) reflected from the observation site just in front of or around the distal end portion 41 of the insertion section 4 forms an image on the light receiving surface (image pickup surface) of the CCD 53 through the objective lens 54. The object image (image light) thus formed is taken by the CCD 53. In other words, the CCD 53 takes the image of the observation site. Taking a specific example, the CCD 53 can take or acquire the object image of at least the epiglottis of a patient and its vicinity at the time when the epiglottis is lifted up by means of the tongue piece 42 of the insertion section 4 and also can take the object image of at least the rima glottidis of a patient and its vicinity (the larynx and the rima glottidis) in the event that the air passage is secured by the insertion section 4.

As the white LED 55 emits a light the light is irradiated on the observation site from the distal end portion 41 of the insertion section 4 through the illumination lens 56, thereby illuminating the observation site. This makes it possible to illuminate the observation site with sufficient brightness.

Although the white LED 55 is single in the illustrated embodiment, two or more white LEDs may alternatively be used depending on the F-number of the objective lens 54 and the sensitivity of the CCD 53.

In this regard, as shown in FIG. 7, the distal end portion of the scope guide bore 44 is slanted toward the intubation tube 200 (the groove 43). In other words, the scope guide bore 44 is inclined with respect to the center line 47 of the distal end portion 41 of the insertion section 4 in such a manner that the centerline 59 of visual field of the CCD (image light acquiring means) 53 of the laryngoscope 5 disposed or received in the scope guide bore 44 can be headed for the intubation tube 200 (the groove 43).

In the intubation operation during which the intubation tube 200 is pushed forward from the distal end portion 41 of the insertion section 4, the intubation tube 200 is adapted to advance toward substantially the center of a screen image displayed by a display 71 described later.

This allows the operator to quite easily ascertain, in the intubation operation, the positional relationship between the rima glottidis, which is an entrance of the trachea, and the distal end portion of the intubation tube 200 with seeing the screen image displayed on the below-mentioned display 71.

The image light acquiring means is not restricted to the configuration set forth above. As an alternative example, the image light acquiring means may be comprised of, e.g., an image guide and a CCD (image pickup device) provided on the proximal end of the image guide. The image guide may include a fiber bundle and an objective lens arranged on the distal end of the fiber bundle, for instance. The fiber bundle is formed by tying together a plurality of individual optical fibers made of, e.g., quartz, multi-component glass, plastics or the like. In this type of image light acquiring means, the image guide picks up the light (image light) reflected from the observation site by use of the objective lens. The image light (object image) thus picked up is transmitted to the CCD via the fiber bundle and then the CCD takes the object image. In this exemplary configuration, the image guide constitutes a means for leading the image light of the observation site (object image) to the image pickup device. The image pickup device may be installed either on the main body 2 or on the intubation assistance instrument 3. In place of the CCD, an eyepiece lens may be employed to enable the operator to observe the observation site with a naked eye.

Likewise, the illumination means is not restricted to the configuration set forth above. As an alternative example, the illumination means may be comprised of, e.g., a light guide and a white LED (light source) provided on the proximal end of the light guide. The light guide may include a fiber bundle and an illumination lens arranged on the distal end of the fiber bundle, for instance. The fiber bundle may be made of the same material as that of the fiber bundle of the image guide noted above. In this type of illumination means, the light guide leads therethrough the light emitted from the white LED and irradiates the light on the observation site at the distal end portion 41 of the insertion section 4, thereby illuminating the observation site. In this exemplary configuration, the light guide constitutes a means (light guide means) for leading the light from the light source to the distal end portion 41 of the insertion section 4. The light source may be installed either on the main body 2 or on the intubation assistance instrument 3.

Formed through the insertion section 4 is an internal bore (lumen) 46 that extends from the proximal end portion to the distal end portion 41, namely, along the longitudinal direction of the insertion section 4. In this connection, the distal end of the internal bore 46 lies on the side of the tongue piece 42 between the groove 43 and the scope guide bore 44, and the proximal end of the internal bore 46 is located on the proximal end portion of the insertion section 4. The internal bore 46 has a cross-section of a generally circular shape.

Inside the internal bore 46, there is provided a suction tube, a forceps or the like not shown in the drawings, for example. The suction tube or the forceps may be detachably fitted into or fixedly secured to the internal bore 46. Use of the suction tube makes it possible to suck up and remove flowable foreign materials such as spit, sputum and so forth. Solid foreign materials can be removed using the forceps.

Although the internal bore 46 is formed in a single number in the illustrated embodiment, two or more internal bores may be provided alternatively.

One or more grooves may also be formed instead of the internal bore 46. As a further alternative, the internal bore 46 and the groove may be respectively provided in a number of one or more.

As shown in FIGS. 1, 2 and 5, the main body 2 is provided with a casing 21 and has a water-proof structure which may be, for example, one of the conventional water-proof structures (configurations) known in the art.

Referring to FIG. 5, the casing 21 includes a case body 22 and an annular coupling portion 23 provided on the distal end side of the case body 22. The case body 22 and the annular coupling portion 23 are fastened to each other by means of bolts (screws) 27. In this case, the gap between the case body 21 and the annular coupling portion 23 is fluid-tightly (air-tightly) sealed with a sealing member (sealing means), e.g. a packing, not shown in the drawings. Alternatively, the case body 21 and the annular coupling portion 23 may be integrally formed with each other.

An annular operating sleeve 24 is provided on the outer circumference of the coupling portion 23 for forward and reverse rotation (in a rotatable manner). Separation of the operating sleeve 24 from the coupling portion 23 is inhibited by means of a retainer member 25. A female thread 241 is formed on the inner circumference of a distal end portion of the operating sleeve 24.

On the outer circumference of the proximal end portion 31 of the intubation assistance instrument 3, there is formed a male thread 311 that threadedly engages with the female thread 241 of the operating sleeve 24.

A groove 231 is formed on the outer circumference of the coupling portion 23 and a sealing member (sealing means) 26 such as a packing or the like is fitted into the groove 231.

In order to attach or affix the intubation assistance instrument 3 to the main body 2, the proximal end portion 31 of the intubation assistance instrument 3 is inserted between the coupling portion 23 of the casing 21 of the main body 2 and the operating sleeve 24, after which the operating sleeve 24 is rotated in a predetermined direction. This enables the intubation assistance instrument 3 to displace in a proximal end direction with respect to the main body 2, whereby the intubation assistance instrument 3 is mounted to the main body 2.

Under the state that the intubation assistance instrument 3 has been mounted to the main body 2, the gap between the coupling portion 23 of the main body 2 and the proximal end portion 31 of the intubation assistance instrument 3 is fluid-tightly (air-tightly) sealed by means of the sealing member 26, which provides fluid-tight (air-tight) sealing to the proximal end side of the scope guide bore 44 of the insertion section 4 of the intubation assistance instrument 3. Thus, the scope guide bore 44 is fluid-tightly (air-tightly) sealed up in its entirety, whereby making it possible to reliably prevent any contamination of the laryngoscope 5 disposed within the scope guide bore 44.

In order to detach the intubation assistance instrument 3 from the main body 2, the operating sleeve 24 is rotated in the direction opposite to the direction described above. This causes the intubation assistance instrument 3 to displace in a distal end direction with respect to the main body 2, whereby the female thread 241 is disengaged from the male thread 311, eventually allowing the intubation assistance instrument 3 to be detached from the main body 2.

If the intubation assistance instrument 3 is detachably mounted to the main body 2 in this manner, it becomes possible to change the intubation assistance instrument 3 each time when the latter is in use and to thereby keep the patient from being infected (secondarily infected) by bacteria, which helps to enhance the safety.

Although the intubation assistance instrument 3 which has been detached may be reused after it is subjected to cleansing, disinfecting and sterilizing processes, it is preferably discarded when used once. This makes it possible to prevent any secondary infection with increased reliability.

The method of attaching or affixing the intubation assistance instrument 3 to the main body 2, i.e., the method of connecting or coupling the intubation assistance instrument 3 and the main body 2 together, is not restricted to the aforementioned one (thread coupling method), but may include a variety of other alternative methods, e.g., a ratchet mechanism method, a bayonet mounting method, a cam method, a locking claw method and a magnetic method.

Referring back to FIGS. 1 and 2, a display (image displaying means) 71 is mounted on the proximal end portion of the main body 2 so as to be rotatable (displaceable) about a shaft 72. In this case, the display 71 maybe designed to be manually rotated by an operator or automatically rotated by the driving power of a drive power source such as an electric motor or the like.

The display 71 is comprised of, e.g., a liquid crystal display device, an organic EL display device or the like and serves to display the image corresponding to the image light acquired by the image light acquiring means, namely, the image (electronic image) of the observation site taken by the CCD 53.

The display 71 is adapted to display a target mark for specifying the location of the rima glottides to easily insert the distal end portion of the intubation tube 200 into the trachea from the rima glottidis, an indicator sign for showing the remaining battery level of a power source described later, a battery warning mark for informing an operator of the battery exchange time when the battery is used up, a lapse time (intubation operation time) counted from the beginning of the intubation operation, which is to avoid the situation that a patient is in an apnea condition for an extended period of time due to a difficulty encountered in the intubation operation, and so forth.

Owing to the fact that the display 71 is rotatable with respect to the main body 2, the display 71 can be oriented in any desired direction regardless of the direction in which the insertion section 4 extends. Accordingly, the image displayed on the display 71 can be readily seen regardless of the posture of a patient or the position of an operator, thus enabling the operator to carry out the intubation operation in an easy and reliable manner.

Furthermore, the display 71 may be configured such that it can be detachably mounted to the main body 2.

A detection means may be additionally provided for detecting the rotation angle (rotation amount) of the display 71 with respect to the main body 2, and the image displayed on the display 71 may be inverted depending on the result of detection.

In addition, the display 71 may be designed to rotate not only in the single axis direction noted above but also in two-axes or three-axes directions, for instance.

The intubation assistance instrument 3 may be rotatable (displaceable) with respect to the main body 2. In this case, the intubation assistance instrument 3 may be designed to be manually rotated by an operator or automatically rotated by the driving power of a drive power means such as an electric motor or the like.

As shown in FIG. 5, a connector portion 61 connected to the connector portion 52 of the laryngoscope 5 is provided in a position inside the main body 2 corresponding to the coupling portion 23. Also provided within the main body 2 are a circuit board connected to the connector portion 61 and the display 71 and a power source and an input/output part connected to the circuit board, which components are not shown in the drawings.

The circuit board includes a LED driving circuit (illuminator driving circuit) for driving the white LED 55, a CCD driving circuit (image pickup device driving circuit) for driving the CCD 53, an image processing circuit for processing the image data outputted from the CCD 53, an image display circuit for converting the image data outputted from the image processing circuit to image data for use in the display 71 and causing an image to be displayed on the display 71, a storage part (storage means) for storing the image data, a central processing circuit and the like.

A battery is removably attached to the power source from which electric power is supplied to various parts including the circuit board.

A lid not shown in the drawings is provided in a predetermined portion of the casing 21 of the main body 2. The lid can be opened to load and unload the battery. In this case, a lock mechanism not illustrated in the drawings is arranged on the lid and the casing 21 to preclude the possibility that the lid is inadvertently or unintentionally opened. The gap between the lid and the casing 21 is fluid-tightly (air-tightly) sealed by means of a sealing member (sealing means) such as a packing or the like not shown in the drawings.

The power source may be configured such that it can be detachably attached to the main body 2. The input/output part includes an external power input terminal through which external power is inputted or supplied, an external monitor output terminal through which the image data is outputted to an external monitor, and an image storing memory terminal to which a memory card (removable memory device) such as a SD card, a CF card or the like is connected.

As illustrated in FIG. 2, a cover 73 is attached to a left side surface of the casing 21 of the main body 2. The cover 73 is opened to gain access to the respective one of the external power input terminal, the external monitor output terminal and the image storing memory terminal. In order to avoid any inadvertent opening or closing, the cover 73 is designed such that it can be opened or closed only with the use of a special tool. The gap between the cover 73 and the casing 21 is fluid-tightly (air-tightly) sealed by means of a sealing member (sealing means) such as a packing or the like not shown in the drawings.

Some or the entirety of the external power input terminal, the external monitor output terminal and the image storing memory terminal may be arranged in, e.g., a battery compartment of the power source.

Description will now be given to one exemplary use (operation) of the intubation assistance apparatus 1.

The intubation assistance apparatus 1 is used in such an instance that a patient has lost consciousness and a need exists to insert the intubation tube 200 into the trachea of the patient.

[1] First, the intubation assistance apparatus 1 is assembled in preparation for insertion of the intubation tube 200.

To this end, the connector portion 52 of the laryngoscope 5 is first connected to the connector portion 61 of the main body 2. If needed, a suction tube, for example, is inserted into and installed within the internal bore 46 of the insertion section 4 of the intubation assistance instrument 3.

Subsequently, the laryngoscope 5 is inserted through the scope guide bore 44 of the insertion section 4 of the intubation assistance instrument 3 and, at the same time, the proximal end portion 31 of the intubation assistance instrument 3 is inserted between the coupling portion 23 and the operating sleeve 24 of the casing 21 of the main body 2. Then, the operating sleeve 24 is rotated in a predetermined direction, thereby attaching the intubation assistance instrument 3 to the main body 2.

[2] Next, the individual parts (the white LED 55, the CCD 53, the display 71 and so forth) of the intubation assistance apparatus I are driven by operating switches not shown in the drawings, and the insertion section 4 of the intubation assistance instrument 3 is pushed into the trachea of the patient through his or her mouth.

More specifically, the insertion section 4 is inserted into the mouth of the patient, while allowing the inner side of the curved portion of the insertion section 4 to extend along the root of the tongue. Seeing and ascertaining the image displayed on the display 71, the operator lifts up the epiglottis of the patient toward the root of the tongue with the tongue piece 42 of the insertion section 4. Then, an appropriate area on the distal end side of the insertion section 4 is brought into contact with the tongue root portion of the patient, thus securing an air passage.

Since the tongue piece 42 (insertion section 4) is optically transparent, the CCD 53 can take an image of the epiglottis through the tongue piece 42 at the time when the epiglottis of the patient is lifted up toward the root of the tongue with the tongue piece 42. The image thus taken is displayed on the display 71. Further, since the tongue piece 42 has a plate-like shape, the epiglottis can be lifted up by means of the tongue piece 42 in an easy, speedy and reliable manner. This makes it possible to secure the air passage in an easy, speedy and reliable fashion.

[3] Once the air passage is secured by the distal end of the insertion section 4, the intubation tube 200 is inserted into the groove 43 from the proximal end portion of the insertion section 4 and continues to be pushed forward. In this process, the intubation tube 200 is guided by the groove 43 and moved forward along the groove 43. Observing the image displayed on the display 71 (including the image of the distal end portion of the intubation tube 200), the operator inserts the distal end portion of the intubation tube 200, which protrudes from the groove 43, into the rima glottidis so that it can reach the trachea.

In this regard, the groove 43 is shaped to ensure that the distal end portion of the intubation tube 200 naturally moves toward the rima glottidis. Thus, the intubation tube 200 is leaded to the rima glottidis by itself.

In this way, the operator can insert the intubation tube 200 from the rima glottidis into the trachea while seeing the image displayed on the display 71, and the intubation tube 200 is naturally leaded to the rima glottidis. Accordingly, it is possible for the operator to insert the intubation tube 200 into the trachea in an easy, speedy and reliable fashion.

[4] Under the state that the intubation tube 200 remains inserted into the trachea, the intubation tube 200 is deformed and detached from the groove 43.

[5] While maintaining this condition, the insertion section 4 is removed or taken out from the mouth of the patient.

In the manner as described above, the intubation tube 200 can be intubated into the trachea of a patient.

As described in the foregoing, the tongue piece 42 is optically transparent in accordance with the intubation assistance apparatus 1. Thus, the epiglottis of a patient can be observed through the tongue piece 42 at the time when the epiglottis is lifted up with the tongue piece 42, thereby making it possible to secure an air passage for the patient in an easy, speedy and reliable manner. Further, owing to the fact that the insertion section 4 of the intubation assistance instrument 3 is provided with the groove 43 for guiding the intubation tube 200, it is possible for the operator to easily and reliably perform the intubation operation of the intubation tube 200. Particularly, since the intubation tube 200 can be guided by the groove 43, it is not necessary for a patient to take a posture even if he suffers from a cervical spine contusion in which the cervical spine of the patient is required to be bent to have the jaws protruded forward during the intubation operation. Accordingly, the intubation operation can be performed easily and reliably even for a patient suffering from a cervical spine contusion (a patient showing the symptoms of intubation trouble).

Since the groove 43 is adapted to detachably or removably hold the intubation tube 200 under the state that the insertion section 4 is inserted through the mouth of the patient, it is possible to, at the completion of the intubation operation, remove the insertion section 4 from the mouth of the patient, with the intubation tube 200 being kept inserted into the trachea of the patient.

Further, provision of the plate-like tongue piece 42 at the distal end portion 41 of the insertion section 4 enables the operator to lift up the epiglottis by use of the tongue piece 42, thereby making it possible to easily and reliably secure an air passage for the patient.

Furthermore, due to the fact that the intubation assistance apparatus 1 is provided with the laryngoscope 5 and the display 7, the operator can observe, e.g., the distal end portion 41 of the insertion section 4, the pharynx and the larynx of a patient, and thus can ascertain the positional relationship between the rima glottidis, and the distal end portion of the intubation tube 200. This allows the intubation operation to be performed easily and reliably.

Moreover, since the intubation assistance instrument 3 is detachably mounted to the main body 2, it becomes possible to change the intubation assistance instrument 3 each time when the latter is in use and to thereby keep the patient from being infected (secondarily infected) by bacteria, which helps to enhance the safety. In particular, the scope guide bore 44 for receiving the laryngoscope 5 is fluid-tightly (air-tightly) sealed under the state that the intubation assistance instrument 3 is mounted to the main body 2. This means that, by changing the intubation assistance instrument 3 every time upon its use, the need to perform cleansing, disinfecting and sterilizing operations can be eliminated so as to reduce the labor hour.

Use of the intubation assistance apparatus 1 makes it possible to shorten the time required in the intubation operation, thus reducing the burden on the patient.

In this regard, the intubation tube 200 is connected at its proximal end to an artificial respiration device which in turn supplies the air into the trachea through the intubation tube 200 inserted into the trachea from the rima glottidis.

While the intubation assistance apparatus of the present invention has been described hereinabove in respect of the illustrated embodiment, this is not intended to limit the scope of the present invention. Instead, each component or element of the intubation assistance apparatus may be replaced with other one that exhibits the same or similar function. Furthermore, other arbitrary components than disclosed above may be added thereto.

For example, the main body 2 may be provided with an electronic data transmission device for transmitting the image data through a telecommunications network to a hospital to which the patient is to be transported. This allows the hospital employees to prepare medical attendance for the patient during the transportation of the patient by an ambulance car.

Furthermore, unlike the above-noted embodiment wherein the intubation assistance instrument 3 is detachably mounted to the main body 2, the intubation assistance instrument 3 may be fixedly secured to the main body 2.

Moreover, the intubation assistance apparatus of the present invention is not limited to the one for use in securing an air passage (for use in inserting an intubation tube into the trachea of a patient). In particular, although the insertion section 4 of the intubation assistance instrument 3 of the present embodiment is inserted into a target area, namely the trachea of a patient or its vicinity thereof, from the mouth of the patient, the insertion section may be inserted into a target area and its vicinity from the mouth cavity or nasal cavity of the patient. Further, the target area is not limited to the trachea, but can be a nasal cavity, pharynx, larynx, esophagus, or the like.

Finally, it is also to be understood that the present disclosure relates to subject matters contained in Japanese Patent Applications Nos. 2005-309150 and 2005-309151 both filed on Oct. 24, 2005 which are expressly incorporated herein by reference in their entireties.

What is claimed is:

1. An intubation assistance apparatus comprising:
a main body;
an intubation assistance instrument provided on the main body, the intubation assistance instrument having an elongated insertion section for insertion into a target site of a patient from a mouth cavity or a nasal cavity of the patient; and
image light acquiring means for acquiring image light of an observation site at a distal end portion of the insertion section,
wherein the intubation assistance instrument includes guide means provided on the insertion section for leading an intubation tube to the target site of the patient when the intubation tube is inserted into the target site, in which the intubation tube is adapted to be removed from the guide means in a state that the insertion section is kept inserted into the target site;
an internal bore in which at least a part of the image light acquiring means is disposed, the internal bore being formed along a longitudinal direction of the insertion section; and
a plate-like protruding portion provided on a distal end portion of the insertion section so as to protrude in a frontward direction.

2. The intubation assistance apparatus as claimed in claim 1, wherein the target site is a trachea or its vicinity of the patient, and the protruding portion has optical transparency.

3. The intubation assistance apparatus as claimed in claim 1, wherein the protruding portion is formed into a generally rectangular shape having a projecting length in the range of 10 to 40 mm.

4. The intubation assistance apparatus as claimed in claim 1, wherein the insertion section is curved at a roughly middle part thereof to have an inner surface at the distal end portion of the insertion section, in which the protruding portion is formed straight to continuously extend from the inner surface of the insertion section.

5. The intubation assistance apparatus as claimed in claim 1, wherein the insertion section has at least one lumen extending along the longitudinal direction of the insertion section for receiving a suction tube or a forceps.

6. The intubation assistance apparatus as claimed in claim 1, wherein the guide means is comprised of a groove extending along the longitudinal direction of the insertion section.

7. The intubation assistance apparatus as claimed in claim 1, wherein the intubation assistance instrument is detachably mounted to the main body.

8. The intubation assistance apparatus as claimed in claim 1, wherein the part of the image light acquiring means disposed in the internal bore is removable from the insertion section.

9. The intubation assistance apparatus as claimed in claim 1, wherein the image light acquiring means has a center line of visual field inclined with respect to a center line of the distal end portion of the insertion section in such a manner as to head for the intubation tube.

10. The intubation assistance apparatus as claimed in claim 1, further comprising image displaying means provided on the main body for displaying an image corresponding to the image light acquired by the image light acquiring means.

11. The intubation assistance apparatus as claimed in claim 10, wherein the intubation assistance apparatus is configured such that, when the intubation tube is pushed forward from the distal end portion of the insertion section, the intubation tube can be moved toward substantially a center of the image displayed on the image displaying means.

12. The intubation assistance apparatus as claimed in claim 1, wherein at least a distal end portion of the internal bore is fluid-tightly sealed by a blockage portion so that at least a distal end portion of the image light acquiring means is not exposed to the outside from the internal bore.

13. The intubation assistance apparatus as claimed in claim 12, wherein the blockage portion has optical transparency.

14. The intubation assistance apparatus as claimed in claim 1, further comprising sealing means for fluid-tightly sealing a gap between the main body and the intubation assistance instrument in a state that the intubation assistance instrument is mounted to the main body wherein a proximal end side of the internal bore is fluid-tightly sealed by means of the sealing means.

15. The intubation assistance apparatus as claimed in claim 14, wherein the internal bore is fluid-tightly sealed in its substantially entirety in a state that the intubation assistance instrument is mounted to the main body.

16. The intubation assistance apparatus as claimed in claim 1, further comprising an illumination means for illuminating the observation site, and at least a part of the illumination means is provided in the insertion section so that it can be removed from the insertion section.

17. The intubation assistance apparatus as claimed in claim 16, wherein the illumination means has a light source provided within the distal end portion of the insertion section.

18. The intubation assistance apparatus as claimed in claim 16, wherein the illumination means includes a light source provided within the main body and light guide means for leading light from the light source to the distal end portion of the insertion section.

19. The intubation assistance apparatus as claimed in claim 16, wherein the part of the image light acquiring means disposed within the internal bore is detachable from the insertion section and a part of the illumination means provided within the insertion section is disposed within the internal bore such that a positional relationship between the part of the illumination means and the part of the image light acquiring means is fixed.

20. In an intubation assistance apparatus comprising a main body, an intubation assistance instrument adapted to be detachably mounted to the main body, the intubation assistance instrument having an elongated insertion section for insertion into a target site of a patient from a mouth cavity or a nasal cavity of the patient; and image light acquiring means for acquiring image light of an observation site at a distal end portion of the insertion section,
wherein the intubation assistance instrument includes guide means provided on the insertion section for leading an intubation tube to the target site of the patient when the intubation tube is inserted into the target site, in which the intubation tube is adapted to be removed from the guide means in a state that the insertion section is kept inserted into the target site;
an internal bore having a disposing potion in which at least a part of the image light acquiring means is adapted to be disposed, the internal bore being formed along a longitudinal direction of the insertion section; and
a plate-like protruding portion provided on a distal end portion of the insertion section so as to protrude in a frontward direction, wherein the protruding portion has optical transparency.
